(19) United States
(12) Patent Application Publication
(10) Pub. No.: US 2025/0255518 A1
(43) Pub. Date: Aug. 14, 2025

(54) SYSTEM AND METHOD FOR USING BLOOD FLOW MEASUREMENTS TO DIAGNOSE AND TREAT HEALTH FUNCTIONS

(71) Applicant: Guy P. Curtis, San Diego, CA (US)

(72) Inventor: Guy P. Curtis, San Diego, CA (US)

(21) Appl. No.: 18/964,380

(22) Filed: Nov. 30, 2024

Related U.S. Application Data
(63) Continuation-in-part of application No. 18/913,138, filed on Oct. 11, 2024.
Application No. 18/913,138 is a continuation-in-part of application No. 18/438,440, filed on Feb. 10, 2024.

(51) Int. Cl. A61B 5/1455; A61B 5/026

(52) U.S. Cl. CPC A61B 5/14551; A61B 5/0261

(57) ABSTRACT

A system and methodology are provided for using a pulse oximeter to monitor a patient's blood flow trends over extended periods of time. For this purpose, changes in oxygen saturation levels which result in infrared color wavelength changes are detected by a pulse oximeter and then compared directly to a base infrared wavelength which was previously correlated with a systolic pressure taken by a sphygmomanometer. Thus, infrared color changes in the patient's blood pressure pulse are used to monitor trends in the patient's blood flow. A correlation factor is established which correlates a predetermined difference between wavelength colors detected by the pulse oximeter with blood pressure measurements taken by a sphygmomanometer. Variations of this correlation factor can then be subsequently monitored independently by the pulse oximeter and compared relative to previously established parameters for blood flow.

This continuation-in-part application claims the benefit of U.S. patent application Ser. No. 18/913,138 filed Oct. 11, 2024, which is a continuation-in-part of U.S. patent application Ser. No. 18/438,440 filed Feb. 10, 2024. The entire contents of application Ser. No. 18/913,138 and application Ser. No. 18/438,440 are hereby incorporated by reference herein.

### FIELD OF THE INVENTION

The present invention pertains generally to systems and methods for noninvasively monitoring a patient's blood flow continuously over an extended period of time. The present invention is particularly, but not exclusively, pertinent to systems and methods which will accurately measure and monitor a patient's blood flow for the purposes of identifying and evaluating the hydrodynamic cardiac and circulatory performance of the patient's vascular system.

### BACKGROUND OF THE INVENTION

It is routine in clinical practice to measure a patient's peak systolic pressure together with his/her comparable diastolic pressure during a heartbeat. A comparison of these two measurements are thereafter typically referred to collectively as the patient's “blood pressure”. Heretofore this “blood pressure” measurement has been considered sufficient for diagnostic purposes. A blood pressure measurement, however, can also be used to trace changes between the amplitudes of a patient's diastolic and systolic pressures during a single heartbeat, to thereby generate a blood flow waveform.

Importantly, the patient's blood flow waveform provides information that is indicative of volumetric blood flow which is also an important diagnostic consideration. The question then is how can a volumetric blood flow value be accurately measured and monitored for clinical purposes. As envisioned by the present invention, oxygen saturation levels which are monitored and measured by a pulse oximeter can be directly correlated to the patient's blood flow waveform for this purpose.

In an evaluation of a patient's heart muscle function, the concept of time requires a dynamic perspective of the blood pressure waveform in a pulse-to-pulse comparison. For instance, time considerations set a patient's pulse rate. Further, a time sequence of pulsatile blood flow waveforms invites dynamic considerations of changes in the extremes of diastolic pressure and systolic pressure. Moreover, the time rate of pressure changes between these pressure extremes during each pulse duration is of diagnostic value. For purposes of this disclosure, all of the variables that are involved in defining a blood pressure waveform are hereinafter collectively referred to as “parametric measurements”.

As recognized for the present invention, an appreciation of the parametric measurements that define a patient's blood flow waveform, and how these parametric measurements change with time in consecutive waveforms of blood flow, can be analyzed in terms of changes in the volume of blood flow as evidenced by the blood flow waveform. This is so because it is the parametric measurements that effectively determine a blood flow waveform. Furthermore, parametric measurements also provide valuable insight into the patient's cardiac performance and peripheral resistance to blood flow in the arteries of the patient.

In a clinical setting, several of the above-mentioned variables can provide valuable long-term information for patients during trauma and/or surgical recovery. The advantage of long-term monitoring of selected measurements is particularly desirable where the information provided would otherwise require repetitive set up activities or additional monitoring equipment. For instance, as noted above, any information regarding blood flow is always a variable of interest. Any one-time measurement of blood flow, however, will not suffice. On the other hand, monitoring trends in blood flow over a longer period of time can be very valuable.

To address the long-term interests for patient recovery. particularly in a clinical setting, it is an object of the present invention to provide continuous information regarding the patient's blood flow. It is another object of the present invention to provide methodological information regarding how a common pulse oximeter can be modified to provide a source of information pertaining to trends in a patient's blood flow. Still another object of the present invention is to provide a system for monitoring a patient's blood flow that is easy to use, is simple to operate and comparatively cost effective.

### SUMMARY OF THE INVENTION

A blood pressure monitor in accordance with the present invention collects parametric measurements from a patient's blood pressure waveform that can be used to assess and evaluate a patient's health condition. Importantly, these parametric measurements are taken from blood pressure values that essentially define a blood flow waveform from a patient. These measurements are then compared with those of both prior and subsequently measured waveforms. This comparison thus provides a basis for a more comprehensive diagnosis of a patient's health condition based on consecutively obtained blood flow pressure measurements.

Structurally, a system for measuring the blood flow of a patient in accordance with the present invention includes a pulse oximeter of any type well known in the pertinent art. The importance here is that it is well known a pulse oximeter will trace changes in blood pressure during a patient's heartbeat. From such a trace the following parametric measurements can be obtained which are of specific importance. These include: 1) changes in diastolic pressure ±Δpd; changes in systolic pressure, ±Δps; and 3) changes in pulse time duration, ±Δtr. Not only are these parametric measurements individually important, the comparisons of these parametric measurements relative to each other, statically and dynamically, are also important. For instance, the ratios of Δpd/Δps, Δps/Δtr, and Δpd/Δtr, as well as the cumulative values ΣΔpd, ΣΔps, and/or ΣΔtr, may be informative insofar as pressure trends are concerned. Further, the time rate of rise from pd to ps during tr is considered relative to the vigor of the heart's contractions, and the slope of the pressure runoff from ps to pd during tr is considered indicative of the peripheral vascular resistance to blood flow. In each case, regardless of whether measurements are considered in a single pulse or in a consecutive pulse-to-pulse context, comparisons of parametric measurements clearly have diagnostic value.

As part of the system for monitoring blood flow, the present invention includes a computer system that receives audiometric signals from the pulse oximeter. Importantly, these signals essentially define the blood flow waveform in the vasculature of the patient. Also included within the computer system is a calculator which uses these parametric measurements from the blood flow waveform to calculate a value for the blood flow volume in the patient's vasculature. Specifically, calculations are made for each consecutive pulse of the patient's heart muscle function. From these calculations, a blood flow volume can be considered comparable to the value of an area bounded by the blood flow waveform and a timeline underneath the blood flow waveform. In this context, for the present invention the timeline is equal in value to the time pulse rate tr of the patient's heart muscle function, e.g. the time between consecutive measurements of diastolic pressures, pd.

Included in the computer system is a monitor that receives information from the calculator to evaluate changes in the parametric measurements of a blood flow wave form. Specifically, by comparing consecutive waveforms, the changes of ±Δpd, ±Δps, and ±Δtr can be determined. Additionally, a video display is provided to present sequential values of the parametric measurements for use in evaluating the patient's health condition.

For an alternative embodiment of the present invention, the difference between raw red and infrared light wavelengths Δλ is measured during each heartbeat. The wavelength difference Δλ can then be correlated to the difference between the diastolic pressure pd and systolic pressure ps for blood pressure Δp. To do this, the present invention relies on the use of a predetermined correlation factor Δλ/Δp. Specifically, the correlation factor correlates oxygen saturation levels of different color wavelength Δλ at the beginning and at the end of each heart beat with blood pressure measurements Δp taken by a blood pressure measuring instrument such as a sphygmomanometer. As appreciated by the present invention, once established, the correlation factor Δλ/Δp is useful to dynamically monitor blood flow.

In detail, the pulse oximeter simultaneously measures oxygen saturation levels based on color frequency differences between wavelengths in both the raw red visible spectrum λrr and in the infrared invisible spectrum λir. In accordance with the present invention, the difference between these wavelengths, Δλ=λir−λrr, is then directly correlated with a previously determined blood pressure measurement Δp=ps−pd.

For this correlation, the value difference between light wavelengths Δλ in a sequence of blood pulses can be considered constant in the correlation factor Δλ/Δp. Preferably, Δλ is determined when a patient is inactive and at rest. Similarly, the value difference between blood pressure measurements Δp is also to be constant, and is preferably determined while the patient is at rest. An important consideration here is that, although Δλ and Δp are considered constant, the predetermined wavelength values λir and λrr as well as the pressure values ps and pd are variable.

Although individual wavelength values λir and λrr, and individual pressure values ps and pd are variable, the static nature of the common correlation factor Δλ/λp allows them to be considered collectively with each other. For example, an alarm can be activated whenever the value of a single individual wavelength value, e.g. a location for λir, differs ± from a predetermined value within a predetermined timeframe. In this example, along with a detected change in λrr, the other variables, λir, ps and pd will also change with the correlation factor Δλ/Δp, because for a one-time calculation of the correlation factor, αλ and Δp are considered constant.

Unlike the finite calculations for many blood characteristics, such as blood pressure p, which is used to assess a patient's health, blood flow factor F is a metric that may require continuous monitoring over an extended period of time. Accordingly, although changes is blood pressure Δp can change dramatically in the short term, changes in the resultant blood flow ΔF may or may not result. Nevertheless, in their relationship with each other, finite blood pressure p and dynamic blood flow F are interactive. Thus, the present invention monitors blood pressure p because it is the motive force that determines blood flow F. The present invention thus uses a pulse oximeter and the correlation of blood oxygen saturation levels with p, for the purpose of monitoring blood flow F.

It is noted here that whenever the maximum infrared wavelength λir in a blood pulse differs ± from a predetermined value or a predetermined location, these variations can be used to give information about blood flow volume. Based on the correlation factor disclosed above, the maximum infrared wavelength λir from a blood pressure measurement can be correlated directly with the systolic pressure ps of the blood pressure measurement. Thus, as long as the correlation factor can be considered constant, subsequent blood pressure measurements of systolic pressures λir including magnitude and location can thereafter be valued directly with blood pressure, without the need for subsequent blood pressure measurements. Consequently, for each subsequent oximeter blood pulse measurement taken by a pulse oximeter, changes in the measured difference between adjacent infrared wavelength ±Δλir of the blood pulse can be considered as being proportional to changes in systolic pressure ±ps.

The dynamic nature of blood flow in the vasculature of a patient is the consequence of many interactive variables. Some of these variables, such as the heart function itself and patient medications have a direct effect on blood flow. Other variables, such as patient activity, and vascular constrictions caused by external pressures can also directly cause changes in blood flow. Furthermore, vascular compromise, e.g. wounds and bruises can have an indirect but, potentially adverse effect on blood flow. Although some of these variables have a short term effect, and others have a long term effect, they all can have a deleterious effect. As disclosed above, between adjacent pulses, λir can somehow change Δλir, with a resultant change in blood pressure. There is then a consequent need to identify an appropriate response.

In accordance with the present invention, a system for monitoring blood flow in the vasculature of a patient involves the following basic components. A device, such as a sphygmomanometer, is required to measure the patient's blood pressure, i.e. a systolic pressure, ps, and a diastolic pressure, pd. Preferably, this blood pressure measurement is taken while the patient is stable while resting in a clinical setting. Next, a device, such as a pulse oximeter, is connected with the patient to measure blood pulse characteristics that include a pulse rate and a maximum oxygen saturation level for each pulse. For purposes of the present invention, a preselected pulse oxygen saturation and its location in a blood pulse, i.e. λir, is correlated with the maximum blood pressure, i.e. systolic pressure, ps, when it first appears.

Based on a correlation factor, as disclosed above, blood pressures can be correlated with magnitude and location of blood oxygen saturation levels. Thus, the present invention monitors blood oxygen saturation levels with an oximeter to determine a maximum blood flow velocity at the measured systolic pressure, ps. A comparator is also included in the system which monitors the sequence of consecutive pules. Specifically, a comparator continuously compares value and location changes in a sum Σ±λir with a premeasured systolic pressure. An evaluator then analyzes a sequence of Σ±λir relative to the measured systolic pressures ps to determined trends in blood flow.

An operational methodology for the present invention will preferably include the following:
Measuring a patient's blood pressure with a sphygmomanometer to determine a diastolic pressure pd and a systolic pressure ps;
Measuring the patient's oxygen saturation level with a pulse oximeter, to determine a pulse rate and preselect reference value, e.g. an infrared wavelength λir in the patient's blood flow;
Correlating ps with λir;
Observing deviations in location and value of the preselected ±Δλir in a sequence of pulses;
Using Δλir as indications of Δps; and
Evaluating Σ±Δλir as an indicator of blood flow trends.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a system for monitoring blood flow in accordance with the present invention, with the system shown operationally connected to a patient;

FIG. 2 is a block diagram of the operative components of the system showing operational interconnections for components of the present invention;

FIG. 3 is a graph showing the pressure variations of an aortic pulse during a heartbeat of the heart muscle function;

FIG. 4 is a depiction of the essential parametric measurements used for describing a pulsed blood flow volume;

FIG. 5 is a line graph showing variations of parametric measurements in a consecutive sequence of pulsed blood flow volumes in the context of a dynamic perspective of blood flow waveforms;

FIG. 6A is a portion of wavelength colors detected by a pulse oximeter which includes raw red light having a wavelength λrr in the visible light spectrum, and infrared light having a wavelength λir in the invisible light spectrum;

FIG. 6B is a line graph showing the relationship between diastolic and systolic pressures in a patient's blood pressure;

FIG. 7 is a graphical presentation of the correlated relationship between changes in blood flow ΔF relative to pressure changes Δp;

FIG. 8 is a line graph representation of dynamic wavelength color variations in a blood pressure pulse;

FIG. 9 is a time line presentation depicting the relationship of wavelength color variations to blood pressure measurements during an operation of the present invention; and

FIG. 10 is a logic flow chart for an operation of the present invention.

### DESCRIPTION OF THE PREFERRED EMBODIMENTS

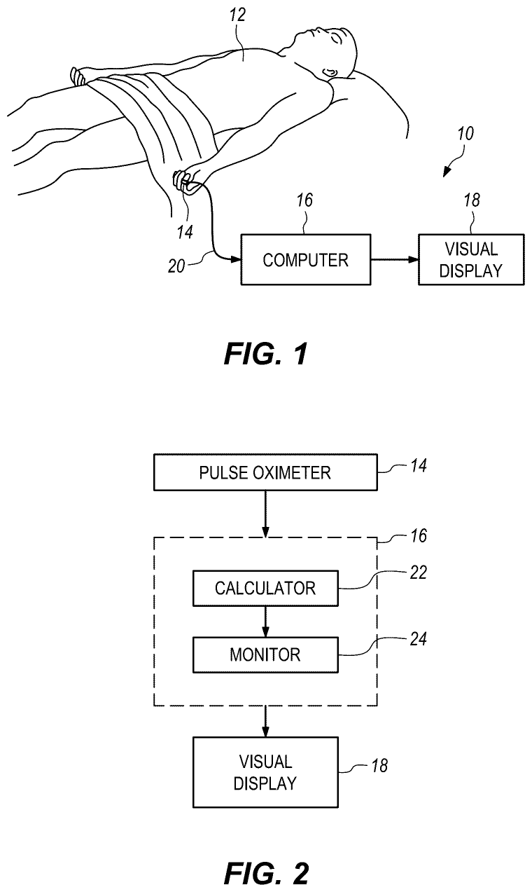

Referring initially to FIG. 1, a system for monitoring blood flow is generally designated 10. In FIG. 1 the system 10 is shown connected to a patient 12 for the purpose of measuring blood flow characteristics of the patient 12. Shown included in the system 10 is a pulse oximeter 14, a computer 16 and a visual display 18. In combination with other components of system 10, the pulse oximeter 14 is shown non-invasively positioned against the patient 12 to receive audiometric signals from the vasculature of the patient 12. Although the pulse oximeter 14 is shown positioned on a finger of the patient 12 in FIG. 1, the present invention envisions that the pulse oximeter 14 may be positioned on the patient 12 wherever positioning is clinically convenient. In any case, system 10 is intended to be electronically engaged with the patient 12 via a connector 20.

In FIG. 2, the computer 16 is shown to include a calculator 22 and a monitor 24. Specifically, calculator 22 is used to receive audiometric signals from the vasculature of patient 12. With these signals values are calculated based blood flow volume characteristics in the vasculature of patient 12. This is done consecutively for each pulse of the patient's heart muscle function. The monitor 24 then evaluates changes in parametric measurements of the blood flow volume as an indicator of the health condition of the patient 12. Results from this evaluation are subsequently transferred to visual display 18 for a presentation of values from the parametric measurements of the blood volume flow are provided. Clinical personnel are thereby provided with the necessary information required to accurately assess a patient's health condition.

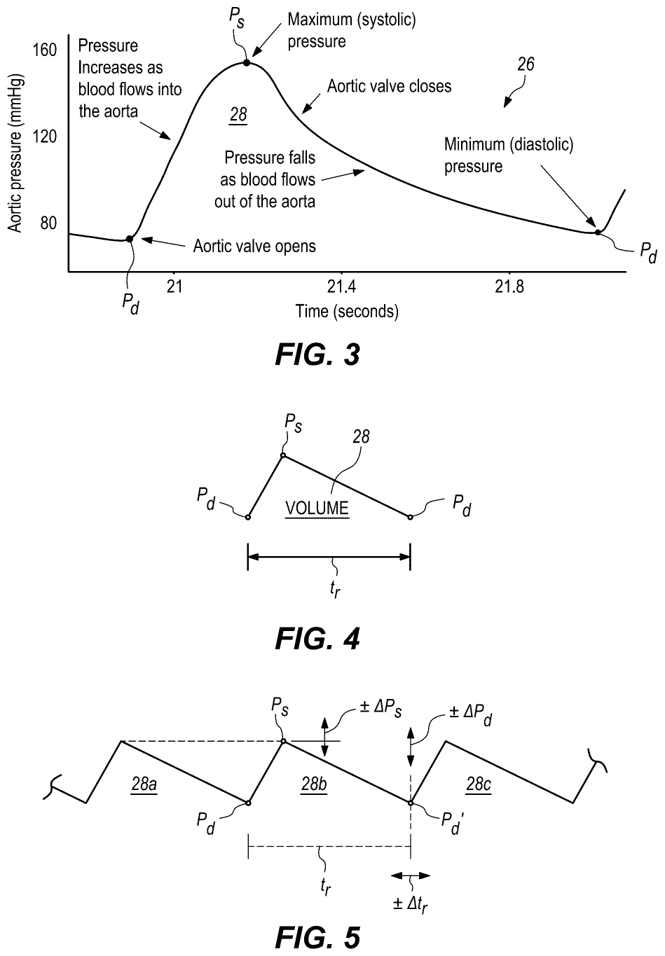

A graph 26 for a generic aortic pulse 28 is shown in FIG. 3 with annotations which illustrate and describe the time variations of aortic activities during the pulse 28. Notably, in FIG. 3 graph 26 indicates that an aortic pulse 28 can be evaluated as pressure changes in a series of connected time segments. More specifically, as shown in FIG. 4, there is a first segment in an aortic pulse 28 that occurs during a pressure rise from a diastolic pressure, pd, to a systolic pressure ps. This first segment is then immediately followed by a second segment that occurs as the pressure falls from the systolic pressure ps to a diastolic pressure pd. At that point, another pulse 28 begins. As shown in FIG. 4, both the first and second segments of an aortic pulse 28 will occur within the pulse duration time of tr.

Further, in FIG. 4 it is to be appreciated that parametric measurements from the pulse oximeter 14 can be taken to define the boundary for a blood flow volume in the vasculature of a patient 12. Specifically, for each pulse 28, the parametric measurements of diastolic pressure, pd, systolic pressure ps, and pulse time duration tr together provide reasonable values for approximating blood flow volume in the heart muscle function of patient 12 (compare FIGS. 3 and 4). It is important here to recognize that for diagnostic purposes, the dynamic values of individual parametric measurements and their variations over time alone provide valuable health information, aside from the actual blood flow volume per se. Note here also that values for the variables pd, ps and tr may vary individually or collectively from pulse to pulse.

As a technical summary for an operation of system 10 of the present invention, FIG. 5 shows a continuous sequence of pulses 28a-c which are provided for the purpose of illustrating variations in the parametric measurements being monitored. As noted above, the individual variables of diastolic pressure, pd, systolic pressure, ps, and pulse duration, tr, can be determined separately for each pulse 28 in the heart function of patient 12. It has also been noted above that each of these parametric measurements can change individually, e.g. from pulse 28a to pulse 28b, et seq. With specific reference to the pulse 28b, note that in comparison with the previous pulse 28a, it is possible that a change of systolic pressure equal to ±Δps may have occurred. Further, it is also noted that during the pulse 28b, the diastolic pressure pd at the beginning of the pulse 28b may change to pd′ at the end of the pulse 28b. Thus, there is a change in pressure equal to ±Δpd during the pulse 28b. It can also happen that the time duration tr will change during between consecutive pulses 28, with an increase or decrease equal to ±Δtr. Accordingly, there are many variations in parametric measurements such as the location of Δλir in the pulse that may have pertinent information for a further analysis of a health condition.

Depending on which aspect of a blood flow waveform is of interest, only certain variables may be important. In the specific case for the present invention, where a volumetric blood flow is of interest and is to be monitored by a pulse oximeter, the important variables are the value and location changes in blood color wavelengths, λ, and blood pressure, p.

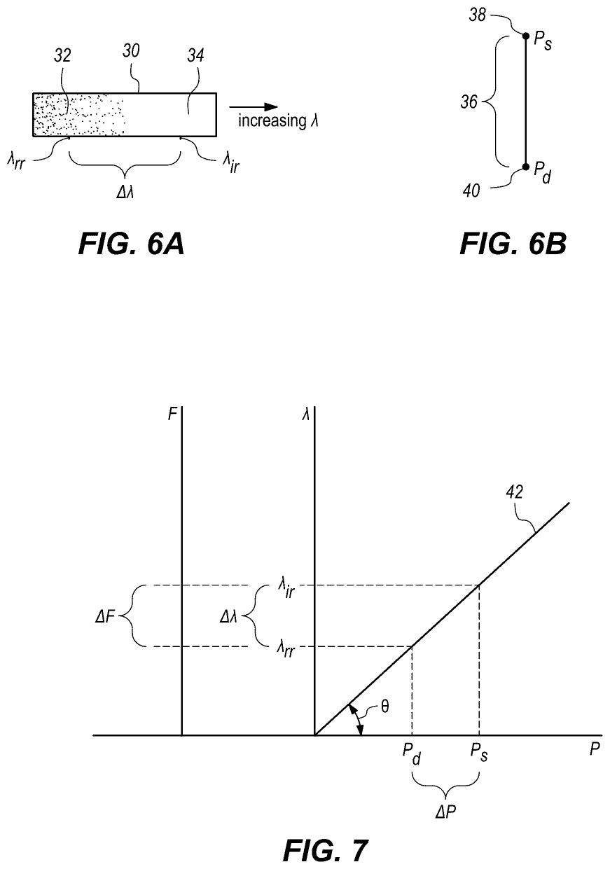

In FIG. 6A, a waveform portion 30 of a blood flow waveform is shown. Within this waveform portion 30 there is a first segment 32 which includes a wavelength λrr of raw red color value which is in the visible light spectrum. In a second segment 34 there is another wavelength λir of infrared color value that is in the invisible light spectrum. For the present invention, both λrr and λir are individually measured by the pulse oximeter 14 relative to a patient's heartbeat. As noted above, however, the wavelength difference between these color segments, i.e. Δλ=λir−λrr, is considered separately as a static constant for successive blood pulses.

In FIG. 6B, a blood pressure measurement Δp 36 is shown which is the difference between a systolic pressure ps 38 and a diastolic pressure pd 40. Like Δλ which is considered as a constant, the blood pressure measurement Δp=ps−pd is also considered constant. Together, Δλ and Δp are considered together in the correlation factor 42 Δλ/Δp.

A correlation factor 42 is provided in FIG. 7 to show how a correlation factor Δλ/Δp compares its components Δλ and Δp, and how changes in blood flow ΔF are affected by changes in pressure Δp in accordance with the correlation factor Δλ/Δp. Graphically, this interaction is shown to be mathematically expressed as an inclination angle Θ, where tan Θ=Δλ/Δp. In any event, the correlation factor Δλ/Δp provides a reference which can be monitored to cause an alarm whenever variations in Δλ and Δp so warrant. As shown in FIG. 7, both changes in Δλ and the value of blood flow change, ΔF, are considered functions of only blood pressure differences Δp, between systolic pressure ps and diastolic pressure pd. As noted above, the variable ΔF is useful for measures of thermodynamic cardiac and circulatory performance.

In summary, the pulse oximeter 14 measures oxygen saturation levels based on a preselected difference in color frequency between raw red wavelength λrr in the visible spectrum, and a wavelength λir the infrared invisible spectrum. In accordance with the present invention, the difference between these wavelengths, Δλ=λir−λrr is directly correlated with a previously determined blood pressure measurement Δp=ps−pd. Moreover, the difference between light wavelengths Δλ and the difference between pressure measurements Δp in a sequence of blood pulses are both considered constant in the correlation factor Δλ/Δp. Further, it is recognized that when a patient is active and is not at rest, although λir and λir are considered relatively constant, individual wavelengths and individual blood pressure measurements Δp can differ considerably, with changes in the location of preselected wavelengths λ in a pulse 28.

As noted above, although individual wavelength values λir and λrr, and individual pressure values ps and pd are variable, the static nature of the common correlation factor Δλ/Δp allows them to be considered directly with each other. For example, an alarm can be activated whenever the value of a single individual wavelength value, e.g. infrared λir, differs ± from a predetermined value within a predetermined timeframe. Moreover, in this example, along with a detected change in λrr, the other variables, λir, ps and pd will also change with the correlation factor Δλ/Δp, because Δλ and Δp are constant. In any event, the correlation factor Δλ/Δp can be continuously recorded and checked by a monitor and alarmed when practicable.

In addition to the information provided above, another measurement of interest which can be derived from characteristics of the vascular system of a patient is the change in his/her blood flow F. The importance of monitoring blood flow F is to provide an early indicator of general systemic issues. As such, blood flow is not just a short-term concern for the volume of blood flow in the vasculature, but also a longer-term concern for vascular functionality. In either case, the focus here is on ± trends in the velocity of the blood flow F. For this purpose, both concerns are systemically interrelated.

As envisioned for the claimed invention, oxygen saturation measurements of a patient's blood are taken with a pulse oximeter 14. From these measurements, a preselected color frequency wavelength λir is used as a reference by which to monitor blood flow F. This use of λir is possible because, in accordance with the correlation factor disclosed above, the color frequency of a wavelength λir from a blood oxygen saturation level measured by the pulse oximeter 14 can be correlated with the systolic pressure ps measured by a blood pressure measuring device, such as a sphygmomanometer. Based on this comparison, and the pulse rate measurement also taken by the oximeter 14, the patient's blood flow can be calculated.

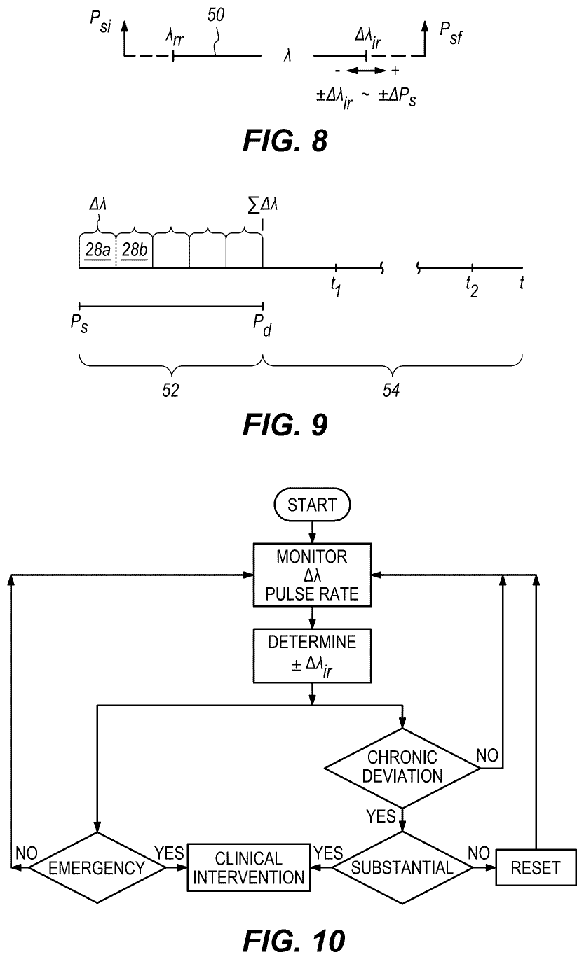

FIG. 8 is a line graph showing a portion 50 of the color spectrum of wavelengths λ in a blood pulse 28. Further, cross referencing FIG. 8 with FIG. 9 specifically shows that the portion 50 extends between an initial systolic pressure psi in the visible wavelengths of raw red color λrr and the following systolic pressure psf in the invisible wavelengths of infrared color λir.

For purposes of this disclosure, the present invention considers only deviations or changes in the location of a preselected infrared wavelength λir within a pulse 28. For its use as a reference point, λir has a unique color frequency bandwidth that is easily detected by a pulse oximeter 14. Further, the preselected infrared wavelength λir is also easily correlated with blood pressure changes in the patient's vasculature. And, λir will appear in each pulse 28.

As shown in FIG. 9, it is an important aspect of the present invention that a preselected λir is detected for each individual pulse 28 during a pressure measurement period 52. For example, FIG. 9 shows a deviation wavelength location ±Δλir can be measured for an exemplary pulse 28a and the next subsequent pulse 28b. Furthermore, the present invention requires a summation of these location changes, Σ±Δλ, during a pressure measurement 52 period. The summation of Σ±Δλ is then to be correlated with blood pressure changes which, in turn, will be used as indicators for blood flow.

As for blood pressure changes Δp, the systolic pressure ps is of particular interest. Specifically, ps is the most easily identifiable pressure in each blood pulse during a pressure measurement period 52. Also, it is the most distinguishable. It can therefore be easily used as a demarcation point in a next blood pulse 28 to end the pressure measurement period 52 and begin the monitoring period 54.

As noted above, an operation of the present invention requires that a summation of infrared color wavelength location deviations Σ±Δλir be made on a pulse-by-pulse basis during each pressure measurement period 52. As indicated in FIG. 9, each pressure measurement period 52 extends between an initial systolic pressure psi in an upstream blood pulse 28 and the following systolic pressure psf in the next successive downstream blood pulse 28. On the other hand, a change in pressure Δps is determined by the change in systolic pressure during a monitoring period 54 which may include a plurality of pressure measurement periods 52. In detail, Δps will be measured as the difference between psi measured at the beginning of a pressure measuring period 52 and a pressure psf measured at the end of the measurement period 52.

The consequence here is that a summation of infrared color frequency movements Σ±Δλir during a single pressure measurement period 52 can thereafter be analytically considered as a functional equivalent of the systolic pressure change Δps during the same pressure measurement period 52 and beyond into the monitoring period 54. Based on this equivalency, the change in systolic pressure Δps is effectively a manifestation of infrared color location deviations Σ±Δλir during the monitoring period 54, which can then be presented by an oximeter as an indicator of blood flow F on a display 56, see FIG. 10.

A methodology for an operation of the system 10 of the present invention is shown in FIG. 10. For an operation of the system 10, two separate functions must be performed. One function, shown by action block 58, is to monitor a preselected infrared color wavelength λir in each successive pulse of the patient's heart beat. This can be done using the pulse oximeter 14. The other function, shown by action block 60, is to measure blood pressure and pulse rate with a blood pressure measuring device, such as a sphygmomanometer.

The calculation block 62, shows that a summation of wavelength changes in blood pulse measurements, Σ±Δλir. is to be determined during the pressure measurement period 52. Also, it is to be appreciated that a patient's blood pressure, including a systolic pressure ps, is also determined during the pressure measurement period 52. However, unlike Σ±Δλir, which continues to be measured continuously during the monitoring period 54, calculation block 64 requires the determination of a change in systolic pressure λps be calculated only with a measurement of ps at the beginning to the pressure measurement period 52, and a measurement of ps at the end of the monitoring period 54.

Decision block 66 in FIG. 10 shows that the summation of wavelength changes Σ±Δλir is to be compared with a predetermined value of Δps to determine whether the summation Σ±Δλir has remained within predetermined guidelines. For example, these guidelines may include whether Σ±Δλir has exceeded the predetermined value for Δps during either the pressure measurement period 52 or the monitoring period 54. Further, a guideline may also be established whereby the summation of wavelength changes Σ±Δλir should never exceed an aggregate value, regardless of time considerations. In any event, the purpose of the present invention is to monitor the summation of wavelength changes Σ±Δλir as an output value provided by the system 10 which can be used to monitor blood flow F for any of a plethora of clinical reasons.

While the system and method for monitoring blood flow in a patient as herein shown and disclosed in detail are fully capable of obtaining the objects and providing the advantages herein before stated, it is to be understood that they are merely illustrative of the presently preferred embodiments of the invention and that no limitations are intended in the details of construction or design herein shown other than as described in the appended claims.

What is claimed is:

1. A system for monitoring blood flow trends in the vasculature of a patient which comprises:
a pulse oximeter for measuring a pulse rate, and for indicating changes in blood oxygen saturation levels within a blood pulse, wherein each pulse has a unique infrared color wavelength λᵢᵣ;
a sphygmomanometer for measuring a blood pressure p of the patient, wherein the pressure measurement includes a systolic pressure pₛ and a diastolic pressure p_{d}, wherein pₛ is an indicator of blood flow;
a monitor for identifying wavelength deviations Δλᵢᵣ, and for summing these deviations Σ±Δλᵢᵣ over a predetermined time period;
a computer for correlating λᵢᵣ with the systolic pressure pₛ; and
a display for presenting the summed wavelength deviations Σ±Δλᵢᵣ, wherein Σ±Δλᵢᵣ is based on values of λᵢᵣ correlated with pressure pₛ, and are used as indications of blood flow trends during the predetermined time period.

2. The system of claim 1 wherein λᵢᵣ is considered as having a constant value in each pulse during a blood pressure measurement.

3. The system of claim 2 wherein λᵢᵣ is arbitrarily taken from the invisible segment of the color spectrum of the blood pulse.

4. The system of claim 3 wherein the number of wavelength deviations summed in a sequence for λ_{ir(base)} is equal to the number of pulses occurring in the blood pressure measurement taken between pₛ and p_{d}.

5. The system of claim 4 wherein wavelength deviations Δλᵢᵣ occur with changes of pₛ. and the location of λᵢᵣ in a pulse.

6. The system of claim 5 wherein pₛ is considered an indicator of blood flow.

7. The system of claim 6 further comprising an alarm feature which is activated whenever Σ±Δλᵢᵣ exceeds a predetermined value within a predetermined time period.

8. The system of claim 7 wherein the predetermined time period is greater than one minute.

9. A system for monitoring blood flow trends in the vasculature of a patient which comprises:
a means for measuring a pulse rate, and changes in blood oxygen saturation levels in a blood pulse;
a means for preselecting a same blood oxygen saturation level for each pulse having a unique infrared color wavelength λᵢᵣ;
a means for measuring a blood pressure p of the patient, wherein the blood pressure measurement includes a systolic pressure pₛ and a diastolic pressure p_{d};
a means for identifying wavelength deviations of ±Δλᵢᵣ between consecutive pulses;
a means for correlating the systolic pressure pₛ with the infrared color wavelength λᵢᵣ;
a means for summing the wavelength deviations Δλᵢᵣ in a sequence of blood pulses to establish a cumulative Σ±Δλᵢᵣ; and
a means for presenting Σ±Δλᵢᵣ as an indicator of blood flow trends.

10. The system of claim 9 wherein λᵢᵣ is considered as having the same value in each pulse during the blood pressure measurement.

11. The system of claim 9 wherein λᵢᵣ is arbitrarily taken for preselection from the invisible segment of the color spectrum of the blood pulse.

12. The system of claim 9 wherein the number of wavelength deviations summed in a sequence of λᵢᵣ is equal to the number of pulses occurring in the blood pressure measurement taken between pₛ and p_{d}.

13. The system of claim 9 wherein pₛ and Δλᵢᵣ are considered as indicators of blood flow.

14. The system of claim 13 wherein Σ±Δλ_{ir(base)} is visually displayed to monitor blood flow trends in the patient's vasculature.

15. The system of claim 9 wherein the means for measuring pulse rate and oxygen saturation levels is an oximeter, wherein the means for measuring blood pressure is a sphygmomanometer, and the means for correlating and summing is a computer.

16. A method for monitoring blood flow trends in the vasculature of a patient which comprises the steps of:
measuring a pulse rate, and changes in blood oxygen saturation levels in a blood pulse;
preselecting a same blood oxygen saturation level for each pulse having a unique infrared color wavelength λᵢᵣ;
measuring a blood pressure p of the patient, wherein the blood pressure measurement includes a systolic pressure pₛ and a diastolic pressure p_{d}, wherein pₛ is considered an indicator of blood flow;
identifying wavelength deviations of ±Δλᵢᵣ between consecutive pulses;
correlating the systolic pressure pₛ with the infrared color wavelength λᵢᵣ;
summing the wavelength deviations Δλᵢᵣ in a sequence of blood pulses to establish a cumulative Σ±Δλᵢᵣ; and
presenting Σ±Δλᵢᵣ as an indicator of blood flow trends.

17. The method of claim 16 wherein λᵢᵣ is considered as having the same value for each pulse during the blood pressure measurement. and wherein λᵢᵣ is arbitrarily taken for preselection from the invisible segment of the color spectrum of the blood pulse.

18. The method of claim 17 wherein the number of wavelength deviations summed in a sequence of λᵢᵣ is equal to the number of pulses occurring in the blood pressure measurement taken between pₛ and p_{d}.

19. The method of claim 18 wherein pₛ and Δλᵢᵣ are considered as an indicator of blood flow.

20. The method of claim 19 wherein Σ±Δλ_{ir(base)} is visually displayed to monitor blood flow trends in the patient's vasculature.
